(19) United States
(12) Patent Application Publication
(10) Pub. No.: US 2026/0165667 A1
(43) Pub. Date: Jun. 18, 2026

(54) X-RAY IMAGING OF A SMALL VOLUME

(71) Applicant: DENTSPLY SIRONA Inc, York, PA (US)

(72) Inventors: Michael Elvers, Bensheim (DE); Johannes Ulrici, Bensheim (DE); Simone Hieber, Baden (CH); Björn Voss, Bensheim (DE); Hong Keun Kim, Oberursel (DE); Konrad Klein, Heidelberg (DE)

(21) Appl. No.: 19/401,055

(22) Filed: Nov. 25, 2025

(30) Foreign Application Priority Data
Dec. 13, 2024 (EP) 24219670.7

(51) Int. Cl. A61B 6/51; A61B 6/00

(52) U.S. Cl. CPC A61B 6/51; A61B 6/4435; A61B 6/542

(57) ABSTRACT

A control unit can receive a digital impression of an oral cavity, receive a selection of a region of interest in the digital impression, determine a size of the region of interest, determine a second reference point in the digital impression, determine a position of a selected region of interest using the first reference point, the second reference point, and the digital impression, control the x-ray detector and the x-ray source to position on opposing sides of the head such that the region of interest can be exposed with an x-ray cone beam, control one or more adjustable shutters to restrict the x-ray cone beam to expose substantially not more than the region of interest, control acquisition of at least one x-ray projection image of the region of interest, and generate an x-ray image using the x-ray projection image or images.

### CLAIM OF PRIORITY

This application claims priority to European Application No. 24219670.7, filed on Dec. 13, 2024, the benefit of priority of which is claimed hereby, and which is incorporated by reference herein in its entirety.

### TECHNICAL FIELD

The present disclosure relates to a system and a method for x-ray imaging in the dental field.

### BACKGROUND

There is ongoing effort to improve x-ray imaging in the dental field.

### BRIEF DESCRIPTION OF THE DRAWINGS

FIG. 1 shows a schematic drawing of an example of an extraoral x-ray system with a CEPH unit.

FIG. 2 shows a schematic drawing of an example of an extraoral x-ray system without a CEPH unit.

FIG. 3 shows a schematic drawing of an example of a computerized imaging/manufacturing system with an intra oral scanner, an MRI device, and subtractive/additive manufacturing devices.

FIG. 4 shows a schematic drawing of an example of a milling machine.

FIG. 5 shows a schematic drawing of an example of a collimator.

FIG. 6 shows another schematic drawing of the example of the collimator.

FIG. 7 shows a schematic drawing of an example of an intra oral scanner, with the dentist acquiring a digital impression of the oral cavity of a patient.

FIG. 8 shows a schematic drawing of an example of the detector when exposed during imaging, wherein the dashed lines show the contour corresponding to the adjusted shutters.

FIG. 9 shows a schematic drawing of an example of a digital impression.

FIG. 10 shows another schematic drawing of the example of the digital impression.

FIG. 11 shows a schematic drawing of an example of the trajectories of the x-ray detector and the x-ray source during imaging.

The reference numbers shown in the drawings designate the elements listed below, which are referred to in the following description of the exemplary examples.
1. Extraoral X-ray system
1′. Imaging System
2. X-ray device
3. X-ray source
4. Primary x-ray detector (PAN/DVT detector)
5. Operating unit (user interface)
6, 6′. Head fixation
7. Bite block
8. Computing unit (Computer)
9. Display device (Screen)
10. Input device (Keyboard)
11. Input device (Mouse)
12. Primary collimator (dashed lines)
13. Cantilever arm
14. Secondary x-ray detector (CEPH detector)
15. Secondary collimator (CEPH collimator)
16. MRI Imaging device
17. 3D Printing unit
18. Post Processing unit
19. Milling machine
20. Dental Block
21. Dental tool
22. Carriage
23. Display
24. Aperture
25. Collimator Blades/shutter
26. Intraoral optical Scanner
DI: Digital Impression
ROI: Region of Interest in DI
P1: First reference point
P2: Second reference point
J: Jawbone of the patient

### DETAILED DESCRIPTION

A cephalometric (CEPH) image (cephalogram), an orthopantomogram (OPG), which is commonly referred to as a panoramic image, and a digital volume tomographic (DVT) image, which is commonly referred to as a 3D image, form part of standard orthodontic diagnostics. These images can provide practitioners with information useful for determining relevant positions in a patient's jaw during the planning, implementation, and follow-up of orthodontic treatment.

In endodontics, for example, selecting a small volume for a 3D image can be particularly beneficial. The 3D images provide practitioners with important information about the position and course of root canals, which can be crucial for successful treatment.

One difficulty encountered when imaging a volume limited to a single tooth lies in the precise positioning of the volume and in the accurate execution of x-ray imaging with such a small volume.

An objective of the present disclosure is to provide a method and system for x-ray imaging of a small volume, specifically a region of interest in a patient's oral cavity.

In one example, a computer-implemented method is used to generate an x-ray image using an x-ray system comprising an x-ray device. The x-ray device includes an x-ray detector, an x-ray source for emitting an x-ray cone beam, and an x-ray collimator having one or more adjustable shutters for setting an aperture for the x-ray radiation. The generated x-ray image is not limited to a specific type and can be a 3D volume image, a tomosynthesis image, or a cephalometric image.

In this example, the method comprises providing a first reference point for positioning a patient's oral cavity and acquiring a digital impression of the patient's oral cavity. A region of interest (ROI), such as a small volume, is selected in the digital impression. The size of the region of interest is determined, and a second reference point is determined in the digital impression. The position of the selected region of interest relative to the x-ray device is determined using the first reference point, the second reference point, and the digital impression. The positions of the x-ray detector and the x-ray source are set on opposing sides of the head of the patient so that the region of interest can be exposed with x-ray cone beam radiation and imaged with the x-ray detector. The shutters are adjusted so that a size of the aperture restricts the x-ray cone beam to expose substantially not more than the region of interest for the set positions of the x-ray detector and the x-ray source. At least one x-ray projection image of the region of interest is acquired at the set positions of the x-ray detector and the x-ray source with the correspondingly adjusted shutters. An x-ray image is generated using the at least one x-ray projection image.

In one example, the tube voltage of the x-ray source and/or the thickness of the shutters (25) are configured such that the shutters do not entirely absorb the x-ray radiation during acquisition of the one or more x-ray projection images. Because the x-ray detector has a large area, the ROI will be imaged more intensely than surrounding dental structures. This configuration allows a practitioner to view the surrounding dental structures of the ROI while preventing unnecessary x-ray exposure to the patient. In this example, the x-ray projection images include a first area showing the selected region of interest and a second area showing an area surrounding the selected region of interest.

In another example, the tube voltage of the x-ray source and/or the thickness of the shutters are configured such that the shutters entirely absorb the x-ray radiation during acquisition of the one or more x-ray projection images. In this example, the x-ray projection images show only the selected region of interest, and unnecessary x-ray exposure to the patient can be prevented to the greatest extent possible.

In another example, during acquisition, the x-ray detector (4) and the x-ray source (3) are moved along a curvilinear trajectory or a circular trajectory. A plurality of x-ray projection images of the region of interest are acquired respectively at a plurality of set positions along the trajectory of the x-ray detector and the x-ray source with the correspondingly adjusted shutters. Using the plurality of x-ray projection images, it is possible to reconstruct a 3D volume image or, alternatively, a panoramic image.

In yet another example, during acquisition, the x-ray detector (4; 14) and/or the x-ray source (3) are moved along a linear trajectory. A plurality of x-ray projection images of the region of interest are acquired respectively at a plurality of set positions along the trajectory of the x-ray detector and the x-ray source with the correspondingly adjusted shutters. Using the plurality of x-ray projection images, it is possible to reconstruct a cephalometric image.

The present disclosure also includes a computer program comprising computer-readable code which, when executed by a computerized x-ray system, causes the system to perform the disclosed method. The computer program can be stored on a storage medium as imaging software.

The present disclosure further includes an extraoral x-ray system comprising an x-ray source (3), an x-ray detector (4; 14), a collimator (12; 15), and a control means for controlling the x-ray source, the x-ray detector, and the x-ray collimator. The control means is configured to perform the disclosed method.

The disclosed method is suitable for reducing the risk of misdiagnosis and incorrect treatment. The method creates diagnostic and therapeutic value for practitioners through more reliable diagnosis.

Various imaging systems (1, 1′) and methods according to the present disclosure will now be explained in greater detail.

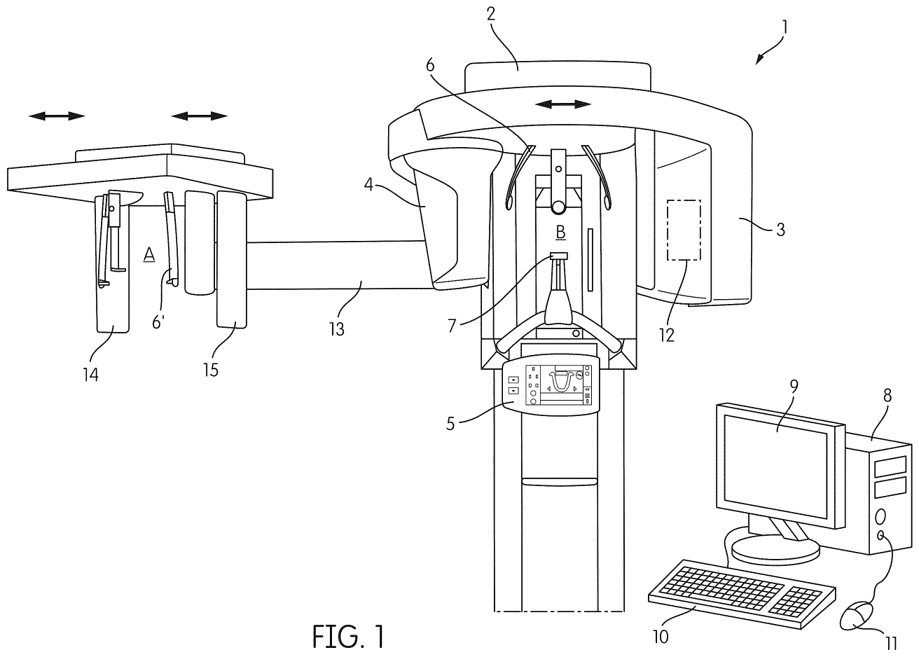

The methods according to the present disclosure, which are described in more detail below, are computer-implemented methods (also referred to herein as software) and can be carried out on computer-assisted systems (1, 1′) as shown in the examples illustrated in FIGS. 1-3. The present disclosure also includes corresponding computer programs (i.e., software) comprising computer-readable code for implementing the methods. The computer program is provided on a non-transitory computer-readable storage medium accessible to the systems (1, 1′).

The computerized extraoral x-ray system (1) shown in FIG. 1 is a multifunctional system for panoramic imaging, digital volume tomographic imaging, and cephalometric imaging. The extraoral x-ray system (1) comprises an x-ray source (3) and a primary x-ray detector (4) that are rotatably arranged around a head of the patient for PAN/DVT imaging (see position B in FIG. 1). The x-ray source (3) and the x-ray detector (4) are mounted on a gantry. The x-ray source (3) includes a primary collimator (12).

The extraoral x-ray system (1) further includes a cantilever arm (13) that holds a secondary x-ray detector (14) (i.e., the ceph detector) and a secondary collimator (15) (i.e., the ceph collimator). In one example, a CMOS-based ceph detector is used. In a further example, the CMOS-based ceph detector comprises a detector with a scintillator or a direct converting detector.

The x-ray ceph detector (14) and the x-ray ceph collimator (15) can be linearly moved along the head of the patient (see position A in FIG. 1). A sequence of projection images of the head of the patient can be obtained by exposing the head of the patient with x-rays from the x-ray source (3) while linearly moving the x-ray ceph detector (14) and the x-ray ceph collimator (15) on opposite sides along the head of the patient with the head positioned between them. Position A in FIG. 1 shows the head of the patient in the ceph mode.

In the ceph mode, the primary x-ray detector (4) is automatically moved out of the x-ray beam path to a side position to avoid blocking x-rays emitted from the x-ray source (3) toward the ceph collimator (15). The extraoral x-ray system (1) includes a control means for energizing the x-ray source (3) and for linearly moving the x-ray ceph detector (14) and the x-ray ceph collimator (15). The head of the patient can be positioned in the x-ray system (1) with a head fixation (6′).

The x-ray ceph collimator (15) comprises a ceph aperture structure. In one example, the x-ray ceph collimator (15) is provided as an adjustable (motorized) multicomponent aperture structure, wherein vertically extending aperture blade components can be moved sideways by a motorized mechanism to adjust the width of the aperture, and wherein horizontally extending aperture blade components can be moved up and down by a motorized mechanism to adjust the height of the aperture. These components can be made from x-ray opaque/absorbing material.

In the PAN/DVT mode, the trajectory of the x-ray source (3) and the x-ray detector (4) during PAN/DVT imaging can describe a circular path. However, the trajectory can also assume a form deviating from a circular path. If several actuators (not shown) are controlled simultaneously, a trajectory deviating from a pure circular path around the head of the patient can be achieved.

The various possible trajectories of the x-ray source (3) and the x-ray detector (4) with respect to the bite block (7) and the head fixation (6) are calibrated for the system (1). The head of the patient can be positioned with a bite block (7) and optionally with a head fixation (6). Position B in FIG. 1 shows the head of the patient in the PAN/DVT mode. The trajectory of the x-ray source (3) and the primary x-ray detector (4) with respect to the bite block (7) and the head fixation (6) are known by the system (1).

The primary x-ray detector (4) detects x-rays emitted by the x-ray source (3) during rotation. X-ray projection images for PAN/DVT imaging are acquired by being read out from the x-ray detector (4). In one example, the primary x-ray detector (PAN/DVT detector) includes a separate PAN detector and a separate DVT detector, which can be rotated around a vertical axis to face the x-ray source (3) in accordance with the respective PAN/DVT imaging mode. In an alternative example, a single flat panel detector capable of both PAN and DVT imaging is used.

Flat panel sensor technologies for x-ray detectors (4) in dental applications are based on either TFT (e.g., amorphous silicon or IGZO) or, depending on size, on one or more CMOS wafers. Cesium iodide (CsI) is commonly used as the scintillator material. The detectors can be operated in 1×1 binning for panorama imaging or, for example, in 2×2 binning for DVT. The readout region of the flat-panel detectors can be predefined (referred to as partial mode). The larger the area to be read out, the lower the resulting frame rate. The dynamic range can be 16 bits. Multiple gain factors can be set and selected depending on the application.

The computerized extraoral x-ray system (1) further comprises an operating unit (5), such as a user interface, for controlling all functionalities of the imaging modes; a computing unit (8), such as a computer; and a display device (9), such as a screen, for visualizing data sets (e.g., projection images and the like) generated by the software. The CEPH/PAN/DVT modes can each be selected by the user through the operating unit (5) and/or the computer unit (8).

The computer may be connected to the extraoral x-ray system (1) via a local area network (not shown) or, alternatively, via the Internet. The computer is connected to input devices such as a keyboard (10), mouse (11), and the like. In some examples, the computer is part of a cloud computing system. In other examples, the computer is integrated into the extraoral x-ray system (1). In yet other examples, all or some of the computations take place in the cloud or on a field programmable gate array (FPGA) implemented in hardware.

The computer executes the computer program and provides data sets, such as cephalometric images, panoramic images, and/or DVT images, for visualization on the screen. The screen may be provided spatially separate from or integrated with the extraoral x-ray system (1). In one example, the computer also controls all functions of the extraoral x-ray system (1). In alternative examples, separate computers are used for control, operation, and image reconstruction.

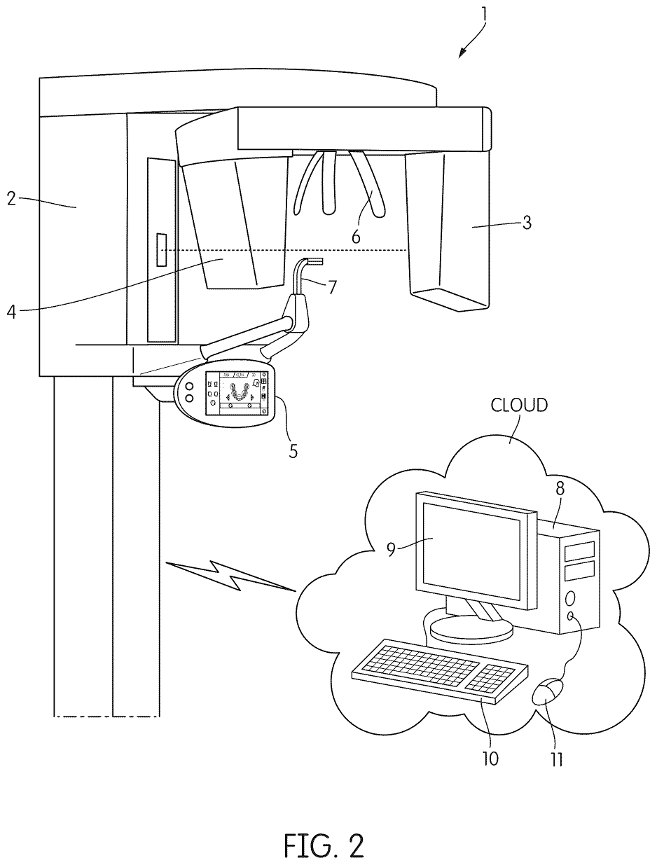

The computerized extraoral x-ray system (1) shown in FIG. 2 is a multifunctional system for panoramic imaging, digital volume tomographic imaging, and virtual cephalometric imaging. The computerized extraoral x-ray system (1) shown in FIG. 2 differs from the x-ray system (1) shown in FIG. 1 in that it lacks a ceph unit attached on a cantilever arm (13). Virtual cephalometric imaging can be performed by back projection with parallel beam geometry using a 3D volume image reconstructed from 2D x-ray projection images acquired during revolution.

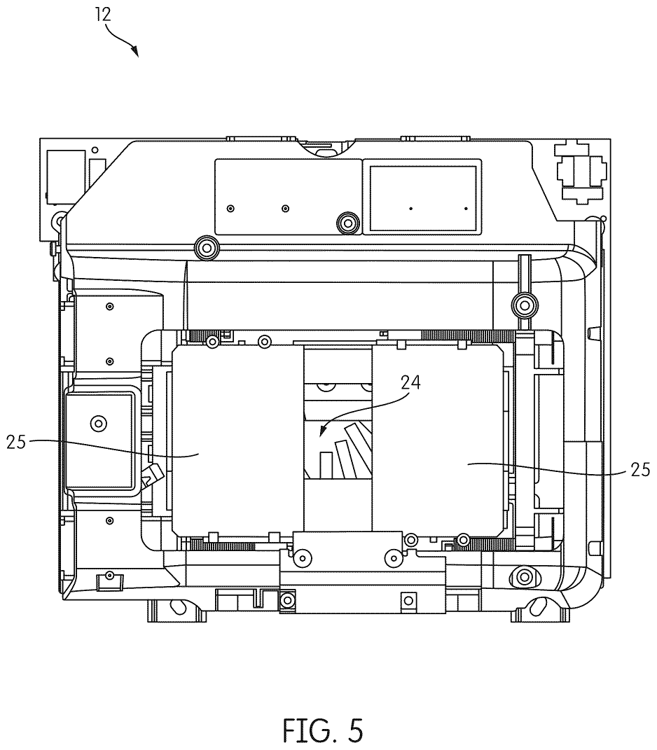

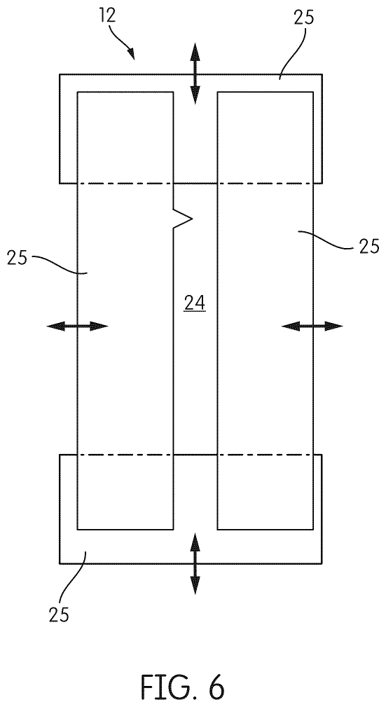

The computerized extraoral x-ray system (1) shown in FIG. 2 includes a primary collimator (shown with dashed lines) (12), which is illustrated in more detail in FIGS. 5 and 6. The x-ray collimator (12) is positioned between the head of the patient and the x-ray source (3). The x-ray collimator (12) is attached to the housing of the x-ray source (3) so that they move and rotate together. The collimator (12) includes an x-ray aperture (24) configured to restrict the x-ray cone beam angle emitted from the x-ray source (3). The width and height of the x-ray aperture (24) are adjustable for varying the x-ray cone beam angle.

In one example, the x-ray collimator (12) is provided as an adjustable (motorized) multicomponent aperture structure, as shown in FIG. 6. In this example, vertically extending aperture blade (25) components can be moved sideways by a motorized mechanism to adjust the width of the aperture (24), and horizontally extending aperture blade (25) components can be moved up and down by a motorized mechanism to adjust the height of the aperture (24). These components can be fabricated from x-ray opaque/absorbing material. The x-ray ceph collimator (15) and the x-ray collimator (12) are similar in structure and functionality.

The 2D x-ray projection images are acquired by being read out from readout regions of the x-ray flat panel detector (4) at a predetermined frame rate. The frame rate can be set and variably controlled by the x-ray system (1).

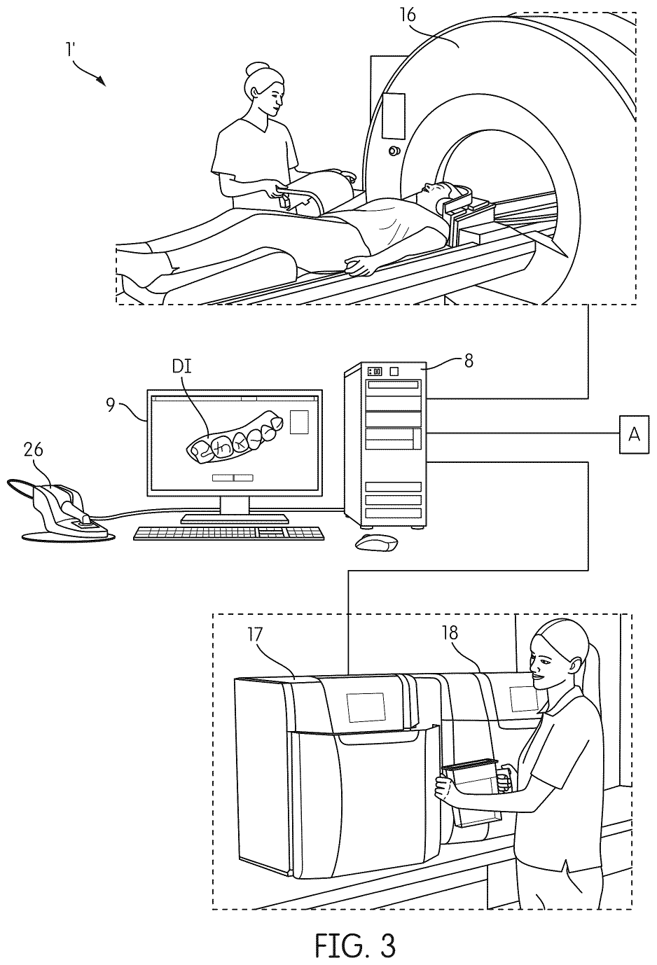

The computerized system (1′) shown in FIG. 3 is a multifunctional system for acquiring 2D images, 3D surface images, and volume images of a patient's dental condition. The system (1′) includes a computer (8), a display (9), and input means such as a keyboard and mouse. The computer (8) can be connected to various imaging devices including a handheld optical intraoral scanner (26), a dental-dedicated magnetic resonance imaging device (16), and an x-ray imaging device (2) with DVT/PAN/CEPH modalities.

In one example, the system (1′) is configurable as an Internet of Things (IOT) system for cloud computing, data exchange, remote control, and similar functions. In this example, the computer (8) can be bidirectionally connected via a local area network and/or the Internet (not shown) to other dental devices such as the optical intraoral scanner (26), the MRI device (16), the x-ray imaging device (2), a dental milling machine (19), a 3D printing unit (17), post-processing units (18), and data storage devices (not shown). The systems (1, 1′) can be used by multiple users, such as dentists.

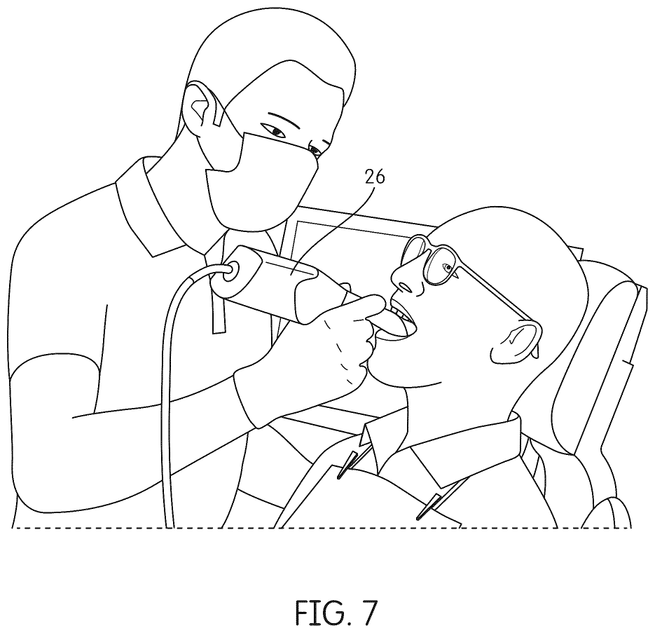

As shown in FIG. 7, the intraoral scanner (26) can be used for acquiring a digital impression (DI) of a patient's oral cavity and dental condition. In some examples, a wireless intraoral scanner can be used and connected to the system via wireless communication. The digital impression comprises a 3D surface image of the dental condition. The digital impression can be used to generate a digital 3D model for designing restoration proposals. Other imaging modalities, such as MR imaging and x-ray imaging, can also be used for generating a digital impression and/or a digital 3D model.

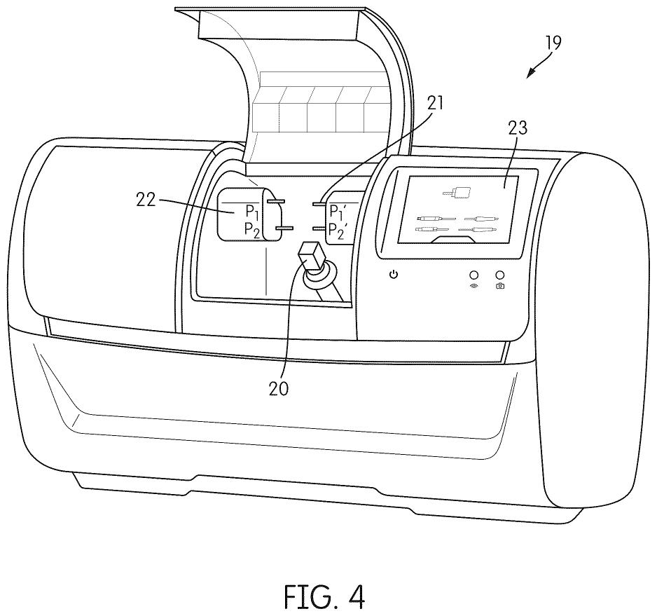

FIG. 4 illustrates a milling machine (19) for manufacturing a dental restoration design. The milling machine (19) comprises a dental blank holder that holds a dental block (20) in a position movable relative to dental tools (21); two driving units/carriages (22), each movably holding one or more dental tools (21) for machining the dental blank (20); and a control unit adapted to control the dental blank holder and the driving units (22) based at least on a trajectory of the dental tools (21) relative to the dental blank (20) and a spatial amount of material removal from the dental blank (20) along the trajectory.

As shown in FIG. 4, the milling machine (19) also includes a user interface (touch screen). Various different dental tools (21) can be used to process the dental blank (20). In one example, the dental tools (21) include micro-RFID tags at their rear side in a housing, which can be recognized by an RFID reader/writer of the milling machine (19).

FIG. 3 also shows a 3D printing unit (3D printer) according to one example. In this example, the 3D printer comprises a resin vat with an at least partially transparent bottom for receiving liquid photoreactive resin for producing a solid component; a building platform for pulling the component layer by layer out from the vat; a projector for projecting layer geometry onto the transparent base; and a transport apparatus for moving the building platform downward or upward in the vat. The 3D printer includes a computer-implemented control unit (not shown) for controlling overall operation.

FIG. 3 also shows a post-processing unit for post-processing operations such as washing, drying, and post-exposure of 3D objects printed by the 3D printer.

In one example, software allows high-quality automatic preparation of 3D printing or milling jobs for dental components such as splints, denture bases, models, and restoration designs such as bridges and crowns.

FIG. 1 shows a schematic drawing of an example of an extraoral x-ray system (1) with a CEPH unit. The extraoral x-ray system (1) is a multifunctional system for panoramic imaging, digital volume tomographic imaging, and cephalometric imaging. The system (1) comprises an x-ray device (2) that includes an x-ray source (3) and a primary x-ray detector (4), which are rotatably arranged around the patient's head for PAN/DVT imaging. The x-ray source (3) and the x-ray detector (4) are mounted on a gantry and the x-ray source (3) has a primary collimator (12), which is shown with dashed lines. The extraoral x-ray system (1) also includes a cantilever arm (13) that holds a secondary x-ray detector (14), which serves as the CEPH detector, and a secondary collimator (15), which serves as the CEPH collimator. The x-ray CEPH detector (14) and the x-ray CEPH collimator (15) can be linearly moved along the patient's head. The system (1) includes a head fixation (6) for positioning the patient's head in the CEPH mode, as shown at position A in the figure. The system (1) also includes a bite block (7) for positioning the patient's head in the PAN/DVT mode, as shown at position B in the figure. The extraoral x-ray system (1) further comprises an operating unit (5), which serves as a user interface for controlling all functionalities of the imaging modes. A computing unit (8), such as a computer, is included for executing the computer program and controlling the system. A display device (9), such as a screen, is provided for visualizing data sets such as projection images, cephalometric images, panoramic images, and DVT images resulting from the software. Input devices including a keyboard (10) and a mouse (11) are connected to the computer (8) to allow user input and control.

FIG. 2 shows a schematic drawing of an example of an extraoral x-ray system (1) without a CEPH unit. The computerized extraoral x-ray system (1) of FIG. 2 is a multifunctional system for panoramic imaging, digital volume tomographic imaging, and virtual cephalometric imaging. The system (1) of FIG. 2 differs from the x-ray system shown in FIG. 2 in that it has no CEPH unit attached on a cantilever arm. The system comprises an x-ray device (2) that includes an x-ray source (3) and a primary x-ray detector (4) that are rotatably arranged around the patient's head. The x-ray source (3) has a primary collimator (12), which is shown with dashed lines and is positioned between the patient's head and the x-ray source (3). The primary collimator (12) is attached into the housing of the x-ray source (3) so that they move and rotate together. The system includes a head fixation (6) and a bite block (7) for positioning the patient's head. The system further comprises an operating unit (5), such as a user interface, for controlling all functionalities of the imaging modes. A computing unit (8), such as a computer, is included, and a display device (9), such as a screen, is provided for visualizing any data sets resulting from the software. Input devices including a keyboard (10) and a mouse (11) are connected to the computer to allow user input. FIG. 2 indicates imaging modes including FAN and CEPH, and shows that the computer may be connected to a CLOUD for cloud computing, data exchange, and remote control.

FIG. 3 shows a schematic drawing of an example of a computerized imaging and manufacturing system (1′). The computerized system (1′) is a multifunctional system for acquiring two-dimensional images, three-dimensional surface images, and volume images of the dental condition of the patient. The system (1′) includes a computer (8) and a display (9) for visualizing data. The computer (8) can be connected to various imaging devices including an intraoral optical scanner (26), a dental dedicated magneto resonance imaging device (16), and an x-ray imaging device (2) with DVT/PAN/CEPH modalities. The system (1′) is preferably configurable as an IoT (internet of things) system for cloud computing, data exchange, and remote control, wherein the computer (8) can be bidirectionally connected via a local area network and/or the internet to other dental devices. The system (1′) also includes a three-dimensional printing unit (17), which serves as a 3D printer for producing dental components. The 3D printing unit (17) comprises a resin vat with an at least partially transparent bottom for receiving liquid photoreactive resin, a building platform for pulling out components layer by layer from the vat, a projector for projecting layer geometry onto the transparent base, and a transport apparatus for moving the building platform downward or upward in the vat. The system (1′) further includes a post processing unit (18) for post processing operations such as washing, drying, and post exposure of the three-dimensional objects printed by the 3D printer. The intraoral scanner (26) is used for acquiring a digital impression DI of the patient's oral cavity and dental condition, wherein the digital impression is a three-dimensional surface image of the dental condition.

FIG. 4 shows a schematic drawing of an example of a milling machine (19) for manufacturing a dental restoration design. The milling machine (19) comprises a dental blank holder which holds a dental block (20) relatively movable with respect to dental tools (21). The milling machine (19) includes two driving units or carriages (22), each movably holding one or more dental tools (21) for machining the dental blank (20). The milling machine (19) includes a control unit adapted to control the dental blank holder and the driving units (22) based at least on a trajectory of the dental tools (21) relative to the dental blank (20) and a spatial amount of material removal from the dental blank (20) along the trajectory. The milling machine (19) also has a user interface in the form of a touch screen display (23) for user interaction and control. Various different dental tools (21) can be used to process the dental blank (20), and the dental tools (21) may have micro-RFID tags at their rear side in a housing which can be recognized by an RFID reader/writer of the milling machine (19).

FIG. 5 shows a schematic drawing of an example of a collimator. The collimator includes an aperture (24) that is formed by adjustable collimator blades (25). The collimator blades (25) can be adjusted to change the size and shape of the aperture (24) through which x-ray radiation passes.

FIG. 6 shows another schematic drawing of the collimator of FIG. 5. The primary collimator (12) is shown with dashed lines in this view. The collimator includes the aperture (24) and multiple collimator blades (25) that can be moved to adjust the size of the aperture (24). The collimator blades (25) include vertically extending aperture blade components that can be moved sideways by a motorized mechanism to adjust the width of the aperture (24) and horizontally extending aperture blade components that can be moved up and down by a motorized mechanism to adjust the height of the aperture (24). The collimator blade components are made from x-ray opaque or absorbing material.

FIG. 7 shows a schematic drawing of an example of an intraoral scanner, while a dentist is acquiring a digital impression of the oral cavity of a patient. The intraoral optical scanner (26) is depicted being used to acquire the digital impression of the patient's oral cavity and dental condition. The digital impression is a three-dimensional surface image of the dental condition.

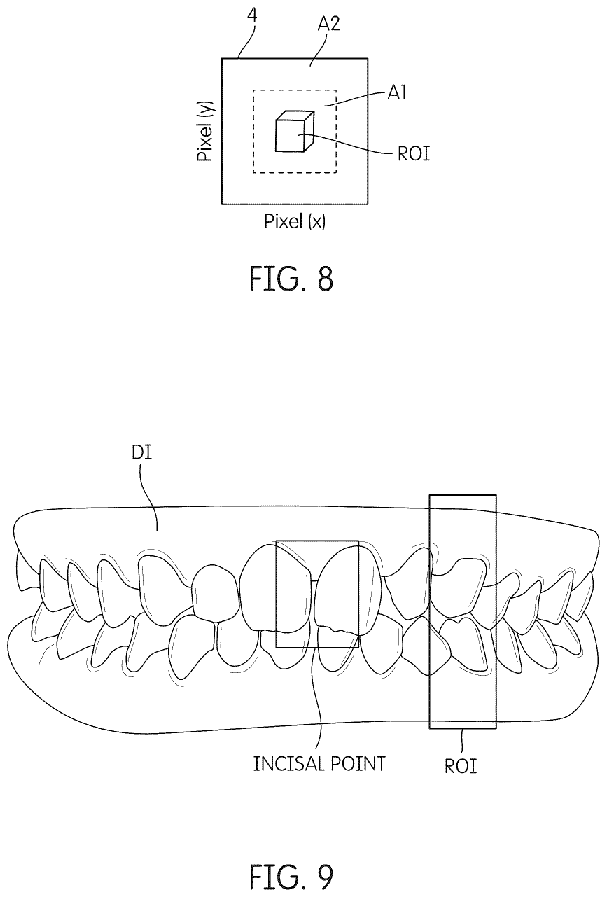

FIG. 8 shows a schematic drawing of an example of the detector when exposed during imaging. The x-ray detector (4) is shown with dashed lines indicating the contour corresponding to the adjusted shutters. The detector includes a first area (A1) showing the selected region of interest (ROI) and a second area (A2) showing an area surrounding the selected region of interest. The figure illustrates the detector with coordinate axes labeled as Pixel (x) and Pixel (y), showing how the region of interest and the surrounding area appear on the detector during exposure.

FIG. 9 shows a schematic drawing of a digital impression (DI) according to an embodiment of the present invention. The digital impression (DI) is a three-dimensional surface image showing the patient's dental arch when viewed from the body axis direction. A region of interest (ROI) is selected within the digital impression (DI). An incisal point is marked on the digital impression (DI) and serves as the second reference point (P2) for determining the position of the selected region of interest relative to the x-ray device.

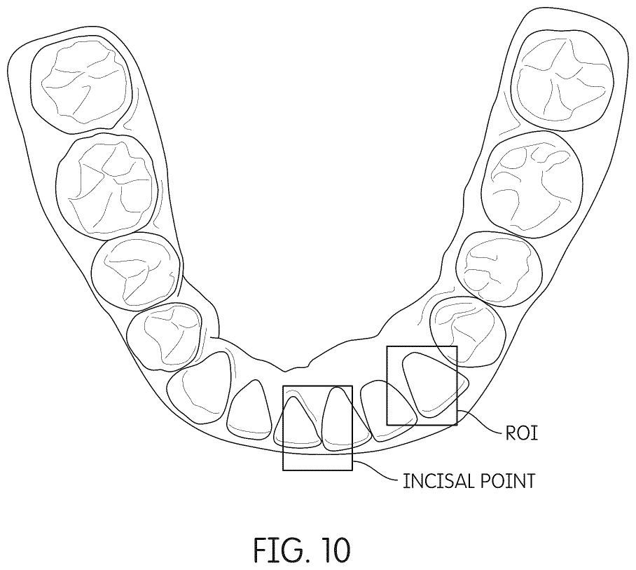

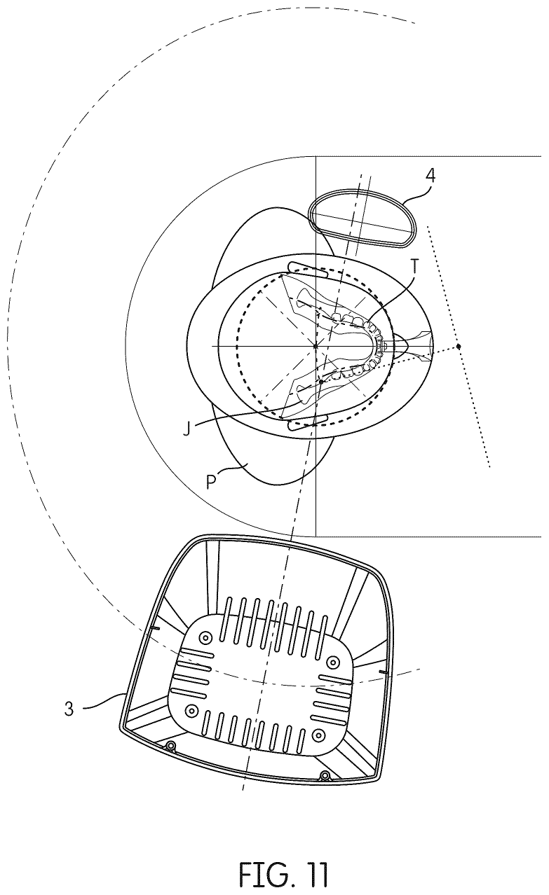

FIG. 10 shows another schematic drawing of the digital impression of FIG. 9. FIG. 10 depicts the digital impression with a region of interest (ROI) marked within the digital impression. The incisal point is shown and serves as a reference point for the positioning calculations. FIG. 10 provides an alternative perspective or configuration of how the region of interest can be defined and positioned within the digital impression.

FIG. 11 shows a schematic drawing of an example of the trajectories of the x-ray detector and the x-ray source during imaging. The x-ray source (3) and the x-ray detector (4) are shown positioned on opposing sides of the jawbone (J) of the patient. The trajectory (T) indicates the path of movement along which the x-ray source (3) and the x-ray detector (4) travel during the acquisition of x-ray projection images. The trajectory (T) can be a curvilinear trajectory, a circular trajectory, or a linear trajectory depending on the imaging mode being performed.

The computer-implemented method for generating an x-ray image of a head of the patient is presently explained in greater detail. The extraoral x-ray system (1, 1′) includes a control means configured to execute the method.

According to the method, the x-ray device (2) includes a first reference point (P1) for positioning a patient's oral cavity. The first reference point (P1) can be established on the bite block (7), as is described in greater detail below. The method comprises the following operations.

In an acquisition operation, a digital impression (DI) of the patient's oral cavity is acquired. This can be accomplished using the intraoral scanner (26). In an alternative example, a digital impression can be retrieved from a database. FIG. 9 shows an exemplary digital impression (DI) comprising 3D data.

In a selection operation, a region of interest (ROI) is selected in the digital impression (DI). The selection can be performed manually by a user via software displayed on the screen (9). Alternatively, automatic selection can be performed via image processing for certain preset anatomical structures, such as teeth.

In a determination operation, the size of the region of interest as selected is determined by the software. This can be calculated from the 3D data of the digital impression.

In a further determination operation, a second reference point (P2) is determined in the digital impression. In one example, this is an incisal point, as shown in FIG. 9. In other examples, other points on the teeth and/or gingiva can be used.

In another determination operation, the position of the selected region of interest is determined relative to the x-ray device (2) using the first reference point, the second reference point, and the digital impression. Because the patient bites the dental block with the incisal point, such calculation can be performed.

In a setting determination operation, the positions of the x-ray detector and the x-ray source on opposing sides of the head of the patient are set so that the region of interest (ROI) can be exposed with x-ray cone beam radiation and imaged with the x-ray detector.

In an adjusting operation, the shutters (25) are adjusted so that a size of the aperture (24) restricts the x-ray cone beam to expose substantially not more than the region of interest (ROI) for the set positions of the x-ray detector and the x-ray source.

In an acquisition operation, at least one x-ray projection image of the region of interest (ROI) is acquired at the set positions of the x-ray detector and the x-ray source with the correspondingly adjusted shutters (25).

In a reconstruction operation, an x-ray image is reconstructed using the at least one x-ray projection image.

In alternative examples, the first reference point (P1) for positioning the patient's oral cavity can be established on the ceph unit via the head fixation (6′) and/or via a laser beam used to locate the patient's oral cavity. In some examples, the ceph unit is provided with a bite block (not shown) to define a first reference point. The positions of the bite blocks are known to the x-ray device (2).

In one example, the tube voltage of the x-ray source and/or the thickness of the shutters (25) are configured such that the shutters do not entirely absorb the x-ray radiation during acquisition of the one or more x-ray projection images. In this example, the x-ray projection images include a first area (A1) showing the selected region of interest and a second area (A2) showing an area surrounding the selected region of interest, as shown in FIG. 8. The second area will appear darker and show surrounding anatomical structures to the practitioner.

In an alternative example, the tube voltage of the x-ray source and/or the thickness of the shutters are configured such that the shutters entirely absorb the x-ray radiation during acquisition of the one or more x-ray projection images. In this example, the x-ray projection images show only the selected region of interest.

In one example, during acquisition, the x-ray detector (4) and the x-ray source (3) are moved along a curvilinear trajectory or a circular trajectory. A plurality of x-ray projection images of the region of interest are acquired respectively at a plurality of set positions along the trajectory of the x-ray detector and the x-ray source with the correspondingly adjusted shutters (25), as described above, so that a size of the aperture (24) restricts the x-ray cone beam to expose substantially not more than the region of interest (ROI) for the set positions of the x-ray detector and the x-ray source.

In an alternative example, during acquisition, the x-ray detector (14) and/or the x-ray source (3) are moved along a linear trajectory. A plurality of x-ray projection images of the region of interest are acquired respectively at a plurality of set positions along the trajectory of the x-ray detector (4) and the x-ray source (3) with the correspondingly adjusted shutters (25), as described above, so that a size of the aperture (24) restricts the x-ray cone beam to expose substantially not more than the region of interest (ROI) for the set positions of the x-ray detector (14) and the x-ray source (3). The linear trajectory can be implemented using the ceph unit. The linear trajectory can also be implemented without the ceph unit by using actuators in the gantry to move the x-ray detector (4) and/or the x-ray source (3) linearly, though movement may be limited depending on the mechanical configuration.

In various examples, the generated x-ray image can be a 3D volume image reconstructed from a plurality of x-ray projection images. In other examples, the generated x-ray image can be a tomosynthesis image, such as a panoramic image, reconstructed from the plurality of x-ray projection images. In still other examples, the generated x-ray image can be a cephalometric image.

What is claimed is:

1. A method of generating an x-ray image using an x-ray system, the x-ray system comprising an x-ray source configured to emit an x-ray cone beam, an x-ray detector, and an x-ray collimator having one or more adjustable shutters, the method comprising:
providing a first reference point for positioning an oral cavity of a patient;
acquiring a digital impression of the oral cavity of the patient;
selecting a region of interest in the digital impression;
determining a size of the region of interest;
determining a second reference point in the digital impression;
determining a position of the selected region of interest relative to the x-ray system using the first reference point, the second reference point, and the digital impression;
setting positions of the x-ray detector and the x-ray source on opposing sides of a head of the patient such that the region of interest can be exposed with the x-ray cone beam and imaged with the x-ray detector;
adjusting the one or more adjustable shutters to restrict the x-ray cone beam to expose substantially not more than the region of interest for the set positions of the x-ray detector and the x-ray source;
acquiring at least one x-ray projection image of the region of interest at the set positions of the x-ray detector and the x-ray source; and
generating an x-ray image using the at least one x-ray projection image.

2. The method of claim 1, wherein:
a tube voltage of the x-ray source and a thickness of the one or more adjustable shutters are configured such that the one or more adjustable shutters do not entirely absorb x-ray radiation during the acquisition of the at least one x-ray projection image; and
the at least one x-ray projection image includes a first area showing the selected region of interest and a second area showing an area surrounding the selected region of interest.

3. The method of claim 1, wherein:
a tube voltage of the x-ray source and a thickness of the one or more adjustable shutters are configured such that the one or more adjustable shutters entirely absorb x-ray radiation outside the region of interest during the acquisition of the at least one x-ray projection image; and
wherein the at least one x-ray projection image shows only the selected region of interest.

4. The method of claim 1, wherein the acquiring at least one x-ray projection image comprises moving the x-ray detector and the x-ray source along a curvilinear trajectory or a circular trajectory and acquiring a plurality of x-ray projection images of the region of interest at a plurality of set positions along the curvilinear trajectory or the circular trajectory.

5. The method of claim 4, wherein a tube voltage of the x-ray source and a thickness of the one or more adjustable shutters are configured such that the one or more adjustable shutters do not entirely absorb x-ray radiation during the acquisition of the plurality of x-ray projection images.

6. The method of claim 4, wherein a tube voltage of the x-ray source and a thickness of the one or more adjustable shutters are configured such that the one or more adjustable shutters entirely absorb x-ray radiation outside the region of interest during the acquisition of the plurality of x-ray projection images.

7. The method of claim 1, wherein the acquiring at least one x-ray projection image comprises moving at least one of the x-ray detector or the x-ray source along a linear trajectory and acquiring a plurality of x-ray projection images of the region of interest at a plurality of set positions along the linear trajectory.

8. The method of claim 7, wherein a tube voltage of the x-ray source and a thickness of the one or more adjustable shutters are configured such that the one or more adjustable shutters do not entirely absorb x-ray radiation during the acquisition of the plurality of x-ray projection images.

9. The method of claim 7, wherein a tube voltage of the x-ray source and a thickness of the one or more adjustable shutters are configured such that the one or more adjustable shutters entirely absorb x-ray radiation outside the region of interest during the acquisition of the plurality of x-ray projection images.

10. The method of claim 1, wherein the x-ray image is a three-dimensional volume image, a tomosynthesis image, or a cephalometric image.

11. An extraoral x-ray system comprising:
an x-ray source configured to emit an x-ray cone beam;
an x-ray detector positioned to receive x-rays from the x-ray source;
an x-ray collimator positioned between the x-ray source and a patient positioning area, the x-ray collimator comprising one or more adjustable shutters configured to set an aperture for the x-ray cone beam;
a positioning mechanism configured to position an oral cavity of the patient using a first reference point;
a control unit operatively connected to the x-ray source, the x-ray detector, and the x-ray collimator, wherein the control unit is configured to:
receive a digital impression of the oral cavity of the patient;
receive a selection of a region of interest in the digital impression;
determine a size of the region of interest;
determine a second reference point in the digital impression;
determine a position of the selected region of interest relative to the x-ray system using the first reference point, the second reference point, and the digital impression;
control the x-ray detector and the x-ray source to position on opposing sides of a head of the patient such that the region of interest can be exposed with the x-ray cone beam;
control the one or more adjustable shutters to restrict the x-ray cone beam to expose substantially not more than the region of interest; and
control acquisition of at least one x-ray projection image of the region of interest and generate an x-ray image using the at least one x-ray projection image.

12. The system of claim 11, wherein a tube voltage of the x-ray source and a thickness of the one or more adjustable shutters are configured such that the one or more adjustable shutters do not entirely absorb x-ray radiation outside the region of interest.

13. The system of claim 11, wherein a tube voltage of the x-ray source and a thickness of the one or more adjustable shutters are configured such that the one or more adjustable shutters entirely absorb x-ray radiation outside the region of interest.

14. The system of claim 11, further comprising a gantry, the x-ray source and the x-ray detector being rotatably mounted on the gantry, the control unit being further configured to control rotation of the x-ray source and the x-ray detector around the head of the patient along a circular or curvilinear trajectory.

15. The system of claim 11, wherein the control unit is further configured to control linear movement of at least one of the x-ray detector or the x-ray source along the head of the patient.

16. The system of claim 11, wherein the x-ray collimator comprises a motorized multicomponent aperture structure including vertically extending aperture blade components movable sideways to adjust a width of the aperture and horizontally extending aperture blade components movable vertically to adjust a height of the aperture.

17. The system of claim 11, wherein:
the positioning mechanism comprises a bite block defining the first reference point; and
a position of the bite block relative to the x-ray detector and the x-ray source is calibrated in the control unit.

18. A non-transitory computer-readable medium storing instructions that, when executed by a processor of an x-ray system, cause the x-ray system to:
receive a digital impression of an oral cavity of a patient;
receive a selection of a region of interest in the digital impression;
determine a size of the region of interest;
determine a second reference point in the digital impression;
determine a position of the selected region of interest relative to the x-ray system using a first reference point associated with a patient positioning mechanism, the second reference point, and the digital impression;
control an x-ray detector and an x-ray source to position on opposing sides of a head of the patient such that the region of interest can be exposed with an x-ray cone beam from the x-ray source;
control one or more adjustable shutters of an x-ray collimator to restrict the x-ray cone beam to expose substantially not more than the region of interest;
control acquisition of at least one x-ray projection image of the region of interest; and
generate an x-ray image using the at least one x-ray projection image.

19. The non-transitory computer-readable medium of claim 18, wherein the instructions further cause the x-ray system to control movement of the x-ray detector and the x-ray source along a curvilinear trajectory or a circular trajectory while acquiring a plurality of x-ray projection images of the region of interest.

20. The non-transitory computer-readable medium of claim 18, wherein the instructions further cause the x-ray system to control movement of at least one of the x-ray detector or the x-ray source along a linear trajectory while acquiring a plurality of x-ray projection images of the region of interest.
